(19) United States
(12) Patent Application Publication
(10) Pub. No.: US 2024/0062848 A1
(43) Pub. Date: Feb. 22, 2024

(54) DETERMINING A DYNAMIC QUALITY METRIC OF A BIOPSY SAMPLE

(71) Applicant: GUARDANT HEALTH, INC., Palo Alto, CA (US)

(72) Inventors: Stephen FAIRCLOUGH, Walnut Creek, CA (US); Aaron Isaac HARDIN, San Francisco, CA (US); David HANNA, Palo Alto, CA (US); Che-yu LEE, Palo Alto, CA (US)

(21) Appl. No.: 18/451,739

(22) Filed: Aug. 17, 2023

Related U.S. Application Data
(60) Provisional application No. 63/371,942, filed on Aug. 19, 2022.

(51) Int. Cl. G16B 20/20; G16B 20/10; G16B 40/20; G06N 5/022

(52) U.S. Cl. CPC G16B 20/20; G16B 20/10; G16B 40/20; G06N 5/022

(57) ABSTRACT

During operation, a computer system may receive information indicating: a number of genetic molecules associated with normal tissue in a sample, and a number of tumor genetic molecules associated with a tumor in the sample. Then, the computer system may determine a sample-specific dynamic quality metric based at least in part on: a type of cancer, sequencing coverage of one or more cancer-specific genomic targets, and a first ratio of the number of tumor genetic molecules to a sum of the number tumor genetic molecules and the number of genetic molecules or a second ratio of the number of tumor genetic molecules to the number of genetic molecules. Next, based at least in part on a comparison of the sample-specific dynamic quality metric and a threshold, the computer system may selectively provide an indication of whether a mutation or the type of cancer is present in the sample.

### CROSS-REFERENCE TO RELATED APPLICATION

This application claims the benefit of U.S. Provisional Patent Application No. 63/371,942, filed Aug. 19, 2022, which is incorporated herein by reference for all purposes.

### FIELD

The described embodiments relate to techniques for assessing the quality of a biopsy sample, such as liquid biopsy or a tissue biopsy sample. Notably, the described embodiments relate to techniques for determining a dynamic quality metric of a biopsy sample.

### BACKGROUND

Cancer is typically caused by the accumulation of mutations within an individual's normal cells. At least some of these mutations disable programmed cell death (apoptosis) and result in improperly regulated cell division. Moreover, the mutations include: single nucleotide variations (SNVs), copy number variations, gene fusions, insertions and deletions (indels), transversions, translocations, and/or inversions.

Cancer is often detected using a tissue biopsy of a tumor followed by analysis of cell pathologies, biomarkers or deoxyribonucleic acid (DNA) extracted from cells in the tissue biopsy sample. In addition, recently it has been proposed that cancer can also be detected using cell-free nucleic acids or cell-free DNA (e.g., circulating nucleic acids, circulating tumor nucleic acids, exosomes, nucleic acids from apoptotic cells and/or necrotic cells) in bodily fluids, such as blood or urine. These so-called ‘liquid biopsy’ tests are non-invasive, can be performed without identifying suspected cancer cells (such as a tumor) to biopsy in advance, and can sample nucleic acids from all parts of a cancer (including a metastasis).

However, liquid biopsies are often complicated by the small amount of nucleic acids released into bodily fluids and the variable recovery of analyzable nucleic acids from these bodily fluids. Therefore, liquid biopsy tests are typically designed to detect very low frequency sequences (e.g., as few as 1 in 1000 molecules at a given locus).

Nonetheless, when operating at or near the edge of the operating window, liquid biopsy test and tissue biopsies can provide incorrect results, such as a false positive or a false negative (e.g., incorrectly detecting a cancer or missing a cancer when it is present). Incorrect results undermine confidence in tissue biopsies and liquid biopsies, and can result in unnecessary or untimely therapeutic interventions, patient suffering and increased patient mortality.

### SUMMARY

In a first group of embodiments, a computer system that determines a sample-specific dynamic quality metric is described. This computer system includes: an interface circuit; a computation device (such as a processor, a graphics processing unit or GPU, etc.) that executes program instructions; and memory that stores the program instructions. During operation, the computer system receives information corresponding to a sample that is associated with a tissue biopsy or a liquid biopsy. Note that the information includes: a number of genetic molecules associated with normal tissue, and a number of tumor genetic molecules associated with a tumor, which may include a number of mutated tumor genetic molecules. (Thus, at least a portion of the tumor genetic molecules contain a sequence variant.) Then, the computer system determines the sample-specific dynamic quality metric based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules. Next, based at least in part on a comparison of the sample-specific dynamic quality metric and a threshold, the computer system selectively provides an indication of whether a mutation or the type of cancer is present in the sample.

Note that the mutated tumor genetic molecules or the mutation may include: an SNV, a copy number variation, a fusion, an insertion, a deletion and/or an epigenetic change. Moreover, the genetic molecules may include DNA.

Furthermore, the computer system may provide one or more (or a combination of) treatment recommendations based at least in part on the indication.

Additionally, the computer system may analyze the sample to determine genetic sequences, epigenetic data and/or a transcriptional state associated with the genetic molecules and the tumor genetic molecules. For example, the analysis may include whole exome sequencing or whole genome sequencing.

In some embodiments, receiving the information corresponding to the sample may include accessing, in the memory, the information corresponding to the sample.

Moreover, the first ratio or the second ratio may include a tumor fraction (which is sometimes referred to ‘tumor purity ’).

Furthermore, the number of tumor genetic molecules in the sample may be based at least in part on histology, liquid biopsy data, pathology information, a simulation, or an output of a pretrained predictive model.

Additionally, the sample-specific dynamic quality metric may be a function of the first ratio or the second ratio when the number of genetic molecules is between 30 and 150.

Note that, when the type of cancer includes lung cancer, the genomic region of interest may include chromosome 7, region p11.2 or a region that includes an epidermal growth factor receptor (EGFR). For example, for non-small cell lung cancer (NSCLC), the genomic regions of interest may include: EGFR exon 19 deletions L858R and T790M, EGFR exon 20 insertions, and/or KRAS G12C. Alternatively or additionally, when the type of cancer includes breast cancer, the genomic region of interest may include chromosome 6, region q25.1-q25.2 or a region that includes an estrogen receptor 1 (ESR1).

In some embodiments, the threshold may be based at least in part on one or more of: the type of cancer, the sequencing coverage of one or more cancer-specific genomic targets, or the first ratio or the second ratio.

Another embodiment provides a computer for use, e.g., in the computer system.

Another embodiment provides a computer-readable storage medium for use with the computer or the computer system. When executed by the computer or the computer system, this computer-readable storage medium causes the computer or the computer system to perform at least some of the aforementioned operations.

Another embodiment provides a method, which may be performed by the computer or the computer system. This method includes at least some of the aforementioned operations.

In a second group of embodiments, a computer system that classifies whether a mutation is present is described. This computer system includes: an interface circuit; a computation device (such as a processor, a graphics processing unit or GPU, etc.) that executes program instructions; and memory that stores the program instructions. During operation, the computer system accesses or determines sequence reads of nucleic acids from a sample or derivatives thereof, wherein the sample comprises a first subset of the nucleic acids derived from tumor cells, a second subset of the nucleic acids derived from non-tumor cells, and the first subset of the nucleic acids includes one or more mutations (which may be at one or more positions or locations in a genome). Note that the determining of the sequence reads may include performing sequencing. Then, the computer system provides a quality threshold (or quality metric) that is a function of a second ratio of the first subset of nucleic acids and the second subset of nucleic acids. Alternatively or additionally, the computer system provides the quality threshold (or quality metric) that is a function of a first ratio of the first subset of nucleic acids and a sum of the first subset of nucleic acids and the second subset of nucleic acids (e.g., expressing the tumor nucleic acids as a proportion of the total nucleic acids instead of the first ratio to the non-tumor nucleic acids). Moreover, the computer system analyzes the sequence reads to classify whether the mutation is present, wherein the classification is based at least in part on the quality threshold.

Note that the sample may be associated with a tissue biopsy or a liquid biopsy.

Furthermore, the analysis may include target areas in DNA, such as disease-specific genomic regions (e.g., regions of the genome that are associated with cancer or other medical conditions, or molecular phenotypes).

Additionally, the classification may be based at least in part on the first ratio, the second ratio and/or genomic locations of the mutations (e.g., sequencing coverage of one or more cancer-specific genomic targets associated with a type of cancer).

In some embodiments, the quality threshold may be determined at one or more genomic locations in the DNA, and the analysis may be performed for the sequence reads at other genomic locations in the DNA.

Moreover, the classification may indicate whether the type of cancer is present.

Note that the mutation may include: an SNV, a copy number variation, a fusion, an insertion, a deletion and/or an epigenetic change.

Furthermore, the computer system may provide one or more (or a combination of) treatment recommendations based at least in part on the classification.

Additionally, the first subset of the nucleic acids may be based at least in part on histology or liquid biopsy data. Note that the quality threshold may be based at least in part on histology, liquid biopsy data, pathology information, a simulation, or an output of a pretrained predictive model.

In some embodiments, the quality threshold may be a function of the first ratio or the second ratio when the number of genetic molecules is between 30 and 150. Moreover, the quality threshold may be reduced as the first ratio or the second ratio is increased.

Note that, when the type of cancer includes lung cancer, the genomic region of interest may include chromosome 7, region p11.2 or a region that includes an EGFR. For example, for non-small cell lung cancer (NSCLC), the genomic regions of interest may include: EGFR exon 19 deletions L858R and T790M, EGFR exon 20 insertions, and/or KRAS G12C. Alternatively or additionally, when the type of cancer includes breast cancer, the genomic region of interest may include chromosome 6, region q25.1-q25.2 or a region that includes an ESR1.

Another embodiment provides a computer for use, e.g., in the computer system.

Another embodiment provides a computer-readable storage medium for use with the computer or the computer system. When executed by the computer or the computer system, this computer-readable storage medium causes the computer or the computer system to perform at least some of the aforementioned operations.

Another embodiment provides a method, which may be performed by the computer or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of a computer system in accordance with an embodiment of the present disclosure.

FIG. 2 is a flow diagram illustrating an example of a method for determining a sample-specific dynamic quality metric using a computer system in FIG. 1 in accordance with an embodiment of the present disclosure.

FIG. 3 is a drawing illustrating an example of communication between components in a computer system in FIG. 1 in accordance with an embodiment of the present disclosure.

FIG. 4 is a drawing illustrating an example of a sample-specific dynamic quality-control threshold in accordance with an embodiment of the present disclosure.

FIG. 5 is a drawing illustrating an example of simulated sensitivity as a function of mutant allele frequency (MAF) in accordance with an embodiment of the present disclosure.

FIG. 6 is a drawing illustrating an example of a tumor-fraction-adjusted threshold in accordance with an embodiment of the present disclosure.

FIG. 7 is a flow diagram illustrating an example of a method for dynamically analyzing a sample in accordance with an embodiment of the present disclosure.

FIG. 8 is a block diagram illustrating an example of a computer in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system (which may include one or more computers) that determines a sample-specific dynamic quality metric of a sample (such as a tissue biopsy sample or a liquid biopsy) is described. This sample-specific dynamic quality metric may indicate whether a test result (such as detecting a type of cancer) is accurate or meets one or more performance metrics. Notably, information corresponding to the sample may include: a number of genetic molecules associated with normal (or non-cancerous) tissue, and a tumor fraction (which specifies a ratio of a number of tumor genetic molecules associated with a tumor to the total number of genetic molecules). For example, the tumor faction may be a ratio of the number of tumor genetic molecules to the total number of genetic molecules at one or more locations or loci, such as the locus with the maximum mutant allele frequency. In some embodiments, the analysis techniques may include embodiments where sequence reads are used (e.g., without molecular counting and, thus, which may represent relative abundance of different sequences) or may include absolute numbers of genetic molecules. Moreover, the computer system may determine the sample-specific dynamic quality metric based at least in part on: a type of cancer and sequencing coverage of one or more cancer-specific genomic targets associated with the type of cancer.

For example, the sample-specific dynamic quality metric may indicate the result is accurate when at least 30-150 genetic molecules are detected. Stated differently, the sample-specific dynamic quality metric may indicate the result is accurate based at least in part on an estimated number of detected mutated tumor genetic molecules, which corresponds to or is a function of the number of genetic molecules in a sample and the tumor fraction. Furthermore, for lung cancer, sequencing coverage of an EGFR may be important, while for breast cancer, sequencing coverage of an ESR1 may be important. Consequently, if the tumor genetic molecules do not spatially cover these regions of the genome for these types of cancer, the results may not be accurate. Next, based at least in part on a comparison of the sample-specific dynamic quality metric and a threshold, the computer system may selectively provide the result, such as an indication of whether a mutation or the type of cancer is present in the sample.

By determining the sample-specific dynamic quality metric, these analysis techniques may reduce the incidence of incorrect results (such as false positives and false negatives) when analyzing samples. In the process, the analysis technique may increase confidence in tissue biopsies and liquid biopsies. Moreover, the analysis techniques may facilitate early detection of cancer, and may provide improved diagnosis, tracking of disease progression and treatment. Furthermore, the analysis techniques may enable further understanding of a variety of types of cancer, and may facilitate the development of new treatments or therapeutic interventions. Consequently, the analysis techniques may reduce unnecessary or untimely therapeutic interventions, patient suffering and patient mortality.

In the discussion that follows, the analysis techniques are used to determine the sample-specific dynamic quality metrics for samples that include or correspond to a wide variety of genetic molecules or information, including: DNA (such as double-stranded or single-stranded), cell-free nucleic acid, ribonucleic acid (RNA), epigenetic information, gene expression or transcriptional state information, protein information, etc. In the discussion that follows, DNA corresponding to at least a portion of an individual's genome is used as an illustrative example.

Moreover, in order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term

As used in this specification and the appended claims, the singular forms ‘a’, ‘an’, and ‘the’ include plural references unless the context clearly dictates otherwise. Thus, e.g., a reference to ‘a method’ includes one or more methods, and/or operations of the type described herein and/or which will become apparent to those persons of ordinary skill in the art upon reading this disclosure and so forth.

Moreover, ‘optional’ or ‘optionally’ means that the subsequently described event or circumstance may or may not occur, and that the description includes cases where said event or circumstance occurs and cases where it does not.

Furthermore, throughout the description and claims of this specification, the word ‘comprise’ and variations of the word, such as ‘comprising’ and ‘comprises,’ means ‘including but not limited to,’ and is not intended to exclude, for example, other components, integers or steps. ‘Exemplary’ means ‘an example of’ and is not intended to convey an indication of a preferred or ideal configuration. ‘Such as’ is not used in a restrictive sense, but for explanatory purposes.

It is also to be understood that the terminology used herein is for the purpose of describing particular embodiments only, and is not intended to be limiting. Furthermore, unless defined otherwise, all technical and scientific terms used herein have the same meaning as commonly understood by one of ordinary skill in the art to which this disclosure pertains. In describing and claiming the methods, computer-readable media, and systems, the following terminology, and grammatical variants thereof, will be used in accordance with the definitions set forth below.

About: As used herein, ‘about’ or ‘approximately’ as applied to one or more values or elements of interest, refers to a value or element that is similar to a stated reference value or element. In certain embodiments, the term ‘about’ or ‘approximately’ refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, ‘adapter’ refers to a short nucleic acid (e.g., less than about 500 nucleotides, less than about 100 nucleotides, or less than about 50 nucleotides in length) that is typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some embodiments, an adapter of the same sequence is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other example embodiments, an adapter is a bell-shaped adapter that includes a blunt or tailed end for joining to a nucleic acid molecule to be analyzed. Other examples of adapters include T-tailed and C-tailed adapters.

Amplify: As used herein, ‘amplify’ or ‘amplification’ in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, ‘barcode’ or ‘molecular barcode’ in the context of nucleic acids refers to a nucleic acid molecule including a sequence that can serve as a molecular identifier. For example, individual ‘barcode’ sequences are typically added to each DNA fragment during next-generation sequencing library preparation so that each read can be identified and sorted before the final data analysis. In some embodiments, the one or more molecular barcodes is at least 2, at least 4, at least 5, at least 6, at least 8, at least 10, at least 15 or at least 20 nucleotides in length. In some embodiments, the polynucleotides of the sample are tagged with at least 5, at least 10, at least 15, at least 20, at least 50, at least 100, at least 500, at least 1000, at least 5000, at least 10.000, at least 50,000 or at least 100,000 different tags/molecular barcodes.

Cancer Type: As used herein, ‘cancer type’ refers to a type or subtype of cancer defined, e.g., by histopathology. Cancer type can be defined by any conventional criterion, such as on the basis of occurrence in a given tissue (e.g., blood cancers, central nervous system or CNS, brain cancers, lung cancers such as small cell and non-small cell, skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers, or another cancer type), unknown primary origin and the like, and/or of the same cell lineage (e.g., carcinoma, sarcoma, lymphoma, cholangiocarcinoma, leukemia, mesothelioma, melanoma, or glioblastoma), and/or cancers exhibiting cancer markers, such as: Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, ‘cell-free nucleic acid’ refers to nucleic acids not contained within or otherwise bound to a cell or, in some embodiments, nucleic acids remaining in a sample following the removal of intact cells. Notably, ‘cell-free nucleic acid’ is ‘cell free’ at the point of isolation from a subject. Therefore, cell-free nucleic acid may not encompass or may be different from isolated cellular DNA. Cell-free nucleic acids can include, e.g., all non-encapsulated nucleic acids sourced from a bodily fluid (e.g., blood, plasma, serum, urine, cerebrospinal fluid or CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell-death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, e.g., a cell-free nucleic acid can be (or a histone associated with the cell-free nucleic acid can be) acetylated, 5-methylated, ubiquitylated, phosphorylated, sumoylated, ribosylated, and/or citrullinated.

Cellular Nucleic Acids: As used herein, ‘cellular nucleic acids’ means nucleic acids that are disposed within one or more cells from which the nucleic acids have originated, at least at the point a sample is taken or collected from a subject, even if those nucleic acids are subsequently removed (e.g., via cell lysis) as part of a given analytical process.

Contamination of samples: As used herein, the terms ‘contamination’ or ‘contamination of samples’ refer to any chemical or digital contamination of one sample with another sample. Contamination can be due to a variety of sources, such as, but not limited to: physical carryover of liquids between samples (e.g., pipetting, automated liquid handling via sample preparation or sequencer systems, manipulating amplified material, etc.), demultiplexing artifacts (e.g., base call errors confounding sample indexes that have limited pairwise Hamming distance, insertion/deletion confounding sample indexes that have limited pairwise edit distance, etc.) and/or reagent impurities (e.g., sample index oligonucleotides contaminated, through either carryover of synthesis errors, with oligonucleotides containing another sample index).

Deoxyribonucleic Acid or Ribonucleic Acid: As used herein, ‘deoxyribonucleic acid’ or ‘DNA’ refers to a natural or modified nucleotide which has a hydrogen group at the 2′-position of the sugar moiety. DNA typically includes a chain of nucleotides including four types of nucleotide bases; adenine (A), thymine (T), cytosine (C), and guanine (G). As used herein, ‘ribonucleic acid’ or ‘RNA’ refers to a natural or modified nucleotide which has a hydroxyl group at the 2′-position of the sugar moiety. RNA typically includes a chain of nucleotides including four types of nucleotide bases; A, uracil (U), G, and C. As used herein, the term ‘nucleotide’ refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, ‘nucleic acid sequencing data,’ ‘nucleic acid sequencing information,’ ‘sequence information,’ ‘nucleic acid sequence,’ ‘nucleotide sequence,’ ‘genomic sequence,’ ‘genetic sequence,’ ‘fragment sequence,’ or ‘nucleic acid sequencing read’ denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Germline Mutation: As used herein, the terms ‘germline mutation’ or ‘germline variation’ are used interchangeably and refer to an inherited mutation (or not one arising post-conception). Germline mutations may be the only mutations that can be passed on to the offspring and may be present in every somatic cell and germline cell in the offspring.

Indel: As used herein, ‘indel’ refers to a mutation that involves the insertion or deletion of nucleotides in the genome of a subject.

Minor Allele Frequency: As used herein, ‘minor allele frequency’ refers to the frequency at which minor alleles (e.g., not the most common allele) occurs in a given population of nucleic acids, such as a sample obtained from a subject. Genetic variants at a low minor allele frequency typically have a relatively low frequency of presence in a sample.

Mutant Allele Fraction: As used herein, ‘mutant allele fraction,’ ‘mutation dose,’ or ‘MAF’ refers to the fraction of nucleic acid molecules harboring an allelic alteration or mutation at a given genomic position/locus in a given sample. MAF is generally expressed as a fraction or a percentage. For example, an MAF of a somatic variant may be less than 0.15.

Mutation: As used herein, ‘mutation’ refers to a variation from a known reference sequence and includes mutations such as, e.g., single nucleotide variants or SNVs, insertions or deletions or indels, and epigenetic alteration. A mutation can be a germline or somatic mutation. In some embodiments, a reference sequence for purposes of comparison is a germline genomic sequence or a matched normal reference of the species of the subject providing a test sample, typically the human genome.

Neoplasm: As used herein, the terms ‘neoplasm’ and ‘tumor’ are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, ‘next generation sequencing’ or ‘NGS’ refers to sequencing technologies having increased throughput as compared to traditional Sanger- and capillary electrophoresis-based approaches, e.g., with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, ‘nucleic acid tag’ refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag includes a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples including nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier or sample identifier). Additionally, or alternatively, nucleic acid tags can be used as molecular barcodes (e.g., to distinguish between different molecules or amplicons of different parent molecules in the same sample or sub-sample). This includes, e.g., uniquely tagging different nucleic acid molecules in a given sample, or non-uniquely tagging such molecules. In the case of non-unique tagging applications, a limited number of tags (such as molecular barcodes) may be used to tag the nucleic acid molecules such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Polynucleotide: As used herein, ‘polynucleotide,’ ‘nucleic acid,’ ‘nucleic acid molecule,’ or ‘oligonucleotide’ refers to a linear polymer of nucleosides (including deoxyribonucleosides, ribonucleosides, or analogs thereof) joined by inter-nucleosidic linkages. Typically, a polynucleotide includes at least three nucleosides. Oligonucleotides often range in size from a few monomeric units, e.g., 3-4, to hundreds of monomeric units. Whenever a polynucleotide is represented by a sequence of letters, such as ‘ATGCCTG,’ it will be understood that the nucleotides are in 5′→3′ order from left to right and that in the case of DNA, ‘A’ denotes deoxyadenosine, ‘C’ denotes deoxycytidine, ‘G’ denotes deoxyguanosine, and ‘T’ denotes deoxythymidine, unless otherwise noted. The letters A, C, G, and T may be used to refer to the bases themselves, to nucleosides, or to nucleotides including the bases, as is standard in the art.

Pretrained Predictive Model: As used herein, a ‘pretrained predictive model’ refers to a numerical model that provide an output (such as a classification or a real-valued numerical value) based at least in part on one or more inputs. The pretrained predictive model is trained using a machine-learning technique (such as a supervised learning technique and/or an unsupervised learning technique, e.g., a clustering technique) and a training dataset. For example, the pretrained predictive model may include a classifier or a regression model that was trained using: a support vector machine technique, a classification and regression tree technique, logistic regression, LASSO, linear regression, a neural network technique (such as a convolutional neural network technique, an autoencoder neural network or another type of neural network technique) and/or another linear or nonlinear supervised-learning technique. Note that the predictive model may be dynamically retrained based at least in part on updates to the training dataset.

Reference Sequence: As used herein, ‘reference sequence’ refers to a known sequence used for purposes of comparison with experimentally determined sequences. For example, a known sequence can be an entire genome, a chromosome, or any segment thereof. A reference typically includes at least about 20, at least about 50, at least about 100, at least about 200, at least about 250, at least about 300, at least about 350, at least about 400, at least about 450, at least about 500, at least about 1000, or more than 1000 nucleotides. A reference sequence can align with a single contiguous sequence of a genome or chromosome or can include non-contiguous segments that align with different regions of a genome or chromosome. Examples of reference sequences include, e.g., human genomes, such as, hG19 and hG38.

Sample: As used herein, ‘sample’ means anything capable of being analyzed by the methods and/or systems disclosed herein.

Sequencing: As used herein, ‘sequencing’ refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, automated capillary-based sequencers, massively parallel sequencing, duplex sequencing, cycle sequencing, single-base extension sequencing, solid-phase sequencing, high-throughput sequencing, massively parallel signature sequencing, emulsion polymerase chain reaction (PCR), co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing (from Illumina of San Diego, California), iSeq 100 (from Illumina of San Diego, California), MiniSeq (from Illumina of San Diego, California), NextSeq (from Illumina of San Diego, California), SOLiD™ sequencing (from Life Technologies, a division of Thermo Fisher Scientific of Waltham, Massachusetts), MS-PET sequencing, and a combination thereof. In some embodiments, sequencing can be performer by a gene analyzer such as, e.g., gene analyzers commercially available from Illumina, Inc., Pacific Biosciences, Inc. (of Menlo Park, California), or Applied Biosystems/Thermo Fisher Scientific, G4 sequencing (from Singular Genomics of La Jolla, California), a flow cell-free sequencing technology such as UG100 (from Ultima Genomics of Newark, California), among many others. Note that, in some embodiments, sequencing may include determining a base identity at a single position or loci.

Sequencing coverage: As used herein, ‘sequence coverage’ refers to sequence reads that are aligned with a particular location or genomic target in the genome. For example, the sequence reads may correspond to a number of genetic molecules (such as nucleic acid molecules) that represent a particular base position.

Sequence Information: As used herein, ‘sequence information’ in the context of a nucleic acid polymer means the order and identity of monomer units (e.g., nucleotides, etc.) in that polymer.

Single Nucleotide Polymorphism: As used herein, the terms ‘single nucleotide polymorphism’ or ‘SNP’ are used interchangeably. They refer to a variation in a single nucleotide that occurs at a specific position in the genome, where each variation is present to some appreciable degree of frequency within a population (e.g., greater than about 1%).

Single Nucleotide Variant: As used herein, ‘single nucleotide variant’ or ‘SNV’ means a mutation or variation in a single nucleotide that occurs at a specific position in the genome.

Somatic Mutation: As used herein, the terms ‘somatic mutation’ or ‘somatic variation’ are used interchangeably. They refer to a mutation in the genome that occurs after conception. Somatic mutations can occur in any cell of the body except germ cells and accordingly, are not passed on to progeny.

Subject: As used herein, ‘subject’ refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms ‘individual’ or ‘patient’ are intended to be interchangeable with ‘subject.’

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Substantially identical: As used herein, the term ‘substantially identical’ refers to two different entities that are 99.9% identical, at least 95% identical, at least 90% identical, at least 85% identical, at least 80% identical, at least 75% identical, at least 70% identical, at least 60% identical or at least 50% identical. In cases where the entity is the molecular barcode, then the term ‘substantially identical’ refers to two different molecular barcodes that have a Hamming distance or edit distance of less than 2, less than 3, less than 4, less than 5, less than 6, less than 7 or less than 8. In cases where the entity is the beginning region or end region, then the term ‘substantially identical’ refers to two different regions that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp or within 25 bp. In cases where the entity is the length of the polynucleotide, then the term ‘substantially identical’ refers to two different lengths that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp, within 25 bp, within 30 bp, within 40 bp or within 50 bp.

Threshold: As used herein, ‘threshold’ refers to a predetermined value used to characterize experimentally determined values of the same parameter for different samples depending on their relation to the threshold. For example, the threshold for the p-value can refer to any predetermined value between 0 and 1 and is used to identify the origin of a nucleic acid variant.

Variant: As used herein, a ‘variant’ can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants, however, are acquired variants and usually have a frequency of less than about 0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

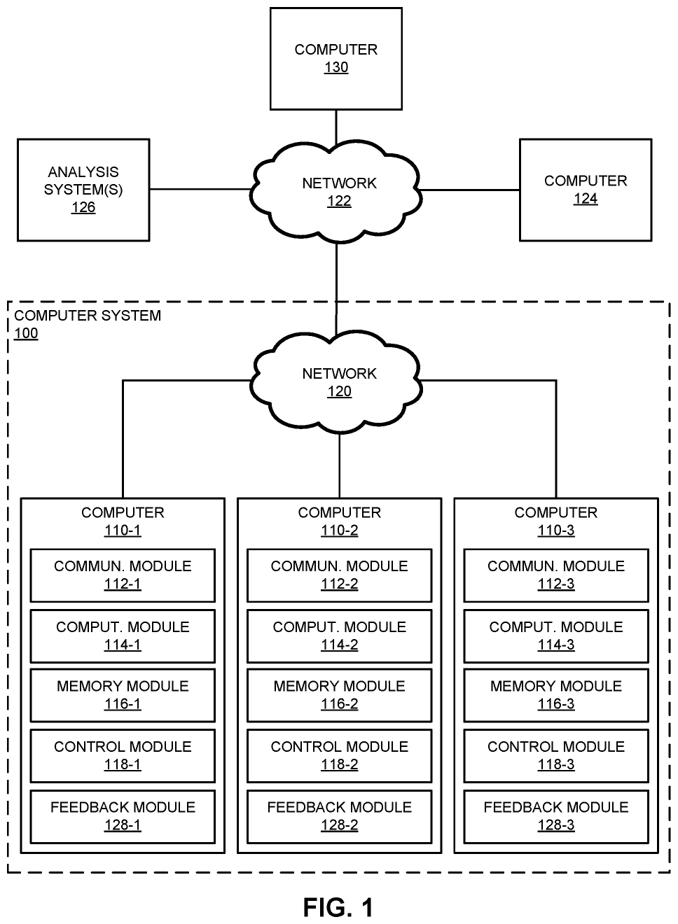

We now describe embodiments of the analysis techniques. FIG. 1 presents a block diagram illustrating an example of a computer system 100. This computer system may include one or more computers 110. These computers may include: communication modules 112, computation modules 114, memory modules 116, and optional control modules 118. Note that a given module or engine may be implemented in hardware and/or in software.

Communication modules 112 may communicate frames or packets with data or information (such as measurement results or control instructions) between computers 110 via a network 120 (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules 112 may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3rd Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies.

In the described embodiments, processing a packet or a frame in a given one of computers 110 (such as computer 110-1) may include: receiving the signals with a packet or the frame; decoding/extracting the packet or the frame from the received signals to acquire the packet or the frame; and processing the packet or the frame to determine information contained in the payload of the packet or the frame. Note that the communication in FIG. 1 may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Note that wireless communication between components in FIG. 1 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. In some embodiments, the communication between the components may use multi-user transmission (such as orthogonal frequency division multiple access or OFDMA).

Moreover, computation modules 114 may perform calculations using: one or more microprocessors, ASICs, microcontrollers, programmable-logic devices, GPUs and/or one or more digital signal processors (DSPs). Note that a given computation component is sometimes referred to as a ‘computation device’.

Furthermore, memory modules 116 may access stored data or information in memory that local in computer system 100 and/or that is remotely located from computer system 100. Notably, in some embodiments, one or more of memory modules 116 may access stored measurement results in the local memory, such as MRI data for one or more individuals (which, for multiple individuals, may include cases and controls or disease and healthy populations). Alternatively or additionally, in other embodiments, one or more memory modules 116 may access, via one or more of communication modules 112, stored measurement results in the remote memory in computer 124, e.g., via network 120 and network 122. Note that network 122 may include: the Internet and/or an intranet. In some embodiments, the measurement results are received from one or more analysis systems 126 (such as PCR, a whole genome sequencer or a partial genome sequencer, e.g., a whole exome sequencer or, more generally, a gene sequencer that uses: a gene sequencing panel, Sanger sequencing, capillary electrophoresis and fragment analysis, microarrays, ligation-based systems, polymerase-based systems, hybridization-based systems, direct or indirect nucleotide identification systems, pyrosequencing, ion- or pH-based detection systems, electronic signature-based systems, next generation sequencing, long-read genetic sequencing, sequencing based on nanopore technology, and/or another sequencing technique) via network 120 and network 122 and one or more of communication modules 112. Thus, in some embodiments at least some of the measurement results may have been received previously and may be stored in memory, while in other embodiments at least some of the measurement results may be received in real-time from the one or more analysis systems 126.

While FIG. 1 illustrates computer system 100 at a particular location, in other embodiments at least a portion of computer system 100 is implemented at more than one location. Thus, in some embodiments, computer system 100 is implemented in a centralized manner, while in other embodiments at least a portion of computer system 100 is implemented in a distributed manner (such as using cloud-computing resources). For example, in some embodiments, the one or more analysis systems 126 may include local hardware and/or software that performs at least some of the operations in the analysis techniques. This remote processing may reduce the amount of data that is communicated via network 120 and network 122. In addition, the remote processing may anonymize the measurement results that are communicated to and analyzed by computer system 100. This capability may help ensure computer system 100 is compatible and compliant with regulations, such as the Health Insurance Portability and Accountability Act, e.g., by removing or obfuscating protected health information in the measurement results.

Although we describe the computation environment shown in FIG. 1 as an example, in alternative embodiments, different numbers or types of components may be present in computer system 100. For example, some embodiments may include more or fewer components, a different component, and/or components may be combined into a single component, and/or a single component may be divided into two or more components.

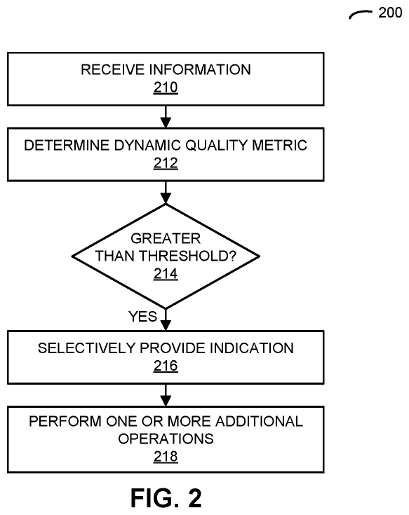

As discussed previously, it is often challenging to accurately detect cancer in a sample. This challenge is exacerbated when the sample is near or at an edge of an operating window of a biopsy technique, such as tissue biopsy or liquid biopsy. Moreover, as described further below with reference to FIGS. 2-7, in order to address these challenges computer system 100 may perform the analysis techniques. Notably, during the analysis techniques, one or more of optional control modules 118 may divide the analysis among computers 110. Then, a given computer (such as computer 110-1) may perform at least a designated portion of the analysis. In particular, computation module 114-1 may receive (e.g., access) information (e.g., using memory module 116-1) specifying genetic molecules (such as at least portions of DNA) in a sample that is associated with a tissue biopsy or a liquid biopsy. For example, the information may be associated with histology or liquid biopsy data. The information may include: a number of genetic molecules associated with normal tissue, and a number of tumor genetic molecules associated with a tumor (which may, equivalently, be specified by a tumor fraction or a ratio of tumor genetic molecules to genetic molecules), which may include a number of mutated tumor genetic molecules. In some embodiments, a number of a given type of genetic molecules may be at one or more locations or loci, as opposed to in the sample as a whole. Note that the number of genetic molecules and the number of tumor genetic molecules are the detected, measured or estimated values of these quantities in the sample. For example, values of the tumor fraction may be estimated by pathologist assessment, as opposed to being directly detected or measured. Alternatively, the tumor fraction may be computed or simulated, e.g., using computer system 100 or a pretrained predictive model that outputs an estimate of the tumor fraction. Furthermore, the mutated tumor genetic molecules may include: an SNV, a copy number variation, a fusion, an insertion, a deletion and/or an epigenetic change.

Then, computation module 114-1 may perform operations in the analysis techniques. Notably, the analysis techniques may include: determining a dynamic quality metric for the sample; and comparing the sample-specific dynamic quality metric to a threshold. The sample-specific dynamic quality metric may be determined based at least in part on: a type of cancer (such as lung cancer or breast cancer), and sequencing coverage of one or more cancer-specific genomic targets associated with the type of cancer. Note that the sequencing coverage of the genomic region of interest may include one or more genes (such as oncogenes) associated with the type of cancer. In particular, when the type of cancer includes lung cancer, the genomic region of interest may include chromosome 7, region p11.2 or a region that includes an EGFR. For example, for non-small cell lung cancer (NSCLC), the genomic regions of interest may include: EGFR exon 19 deletions L858R and T790M, EGFR exon 20 insertions, and/or KRAS G12C. Alternatively or additionally, when the type of cancer includes breast cancer, the genomic region of interest may include chromosome 6, region q25.1-q25.2 or a region that includes an ESR1.

For example, test results on the sample may meet one or more desired performance metrics (such as an accuracy, a confidence, a sensitivity and/or a specificity greater than 80%, 85%, 90%, 95% or 98%) based at least in part on or a function of the tumor fraction (such as a first ratio of the number of tumor genetic molecules to a sum of the number tumor genetic molecules and the number of genetic molecules or a second ratio of the number of tumor genetic molecules to the number of genetic molecules). Notably, because the number of mutated tumor genetic molecules is a fraction of the number of tumor genetic molecules, the sample-specific dynamic quality metric may be a function of the first ratio or the second ratio when the number of genetic molecules is between 30 and 150. Below 30 genetic molecules the sample may be of insufficient size and test results for the sample may not meet the one or more performance metrics (e.g., because there may be insufficient tumor genetic molecules in the sample), while above 30 tumor genetic molecules there may be of sufficient size and the test results may meet the one or more performance metrics (such as sufficient sensitivity). Moreover, between 30 and 150 genetic molecules, the one or more performance metrics for the test results may depend on multiple factors, as indicated by the comparison of the sample-specific dynamic quality metric and the threshold. In some embodiments, the threshold may be based at least in part on one or more of: the type of cancer, the sequencing coverage of one or more cancer-specific genomic targets, and/or the tumor fraction. Note that the aforementioned number of tumor genetic molecules, the number of mutated tumor genetic molecules, the number of non-tumor genetic molecules and/or the number of genetic molecules may be what is detected, as opposed to what is in the sample (which may include undetected genetic molecules).

In some embodiments, the analysis techniques may be performed using a look-up table. For example, values of the quality metric and/or the threshold may be stored in memory module 116-1 as a function of the type of cancer, the number of tumor genetic molecules, the tumor fraction and/or the sequencing coverage. Alternatively or additionally, the analysis techniques may be performed using a pretrained predictive model, such as a classifier or a regression model. Notably, the type of cancer, the number of tumor genetic molecules, the tumor fraction and/or the sequencing coverage may be input to the pretrained predictive model, and the pretrained predictive model may output the sample-specific dynamic quality metric, the threshold, and/or a result of the comparison. In general, the pretrained predictive model may include a machine-learning model or a neural network, which was previously trained using a training dataset. Note that the neural network may include or combine: one or more convolutional layers, one or more residual layers and one or more dense or fully connected layers, and where a given node in a given layer in the given neural network may include an activation function, such as: a rectified linear activation function or ReLU, a leaky ReLU, an exponential linear unit or ELU activation function, a parametric ReLU, a tanh activation function, and/or a sigmoid activation function.

After performing at least some of the operations in the analysis techniques, computation module 114-1 may selectively output or provide information specifying or corresponding to the test results on the sample. For example, when the sample-specific dynamic quality metric exceeds the threshold (indicating that the sample size is sufficient and the test results are considered to meet the one or more performance metrics), computation module 114-1 may access (in memory module 116-1) and then selectively output or provide the information. Note that the information may include a cancer classification, such as an indication of whether a mutation or the type of cancer is present in the sample (e.g., that a clinical variant has been detected). Alternatively or additionally, the information may include one or more (or a combination of) treatment recommendations (such as a recommendation for radiation or chemotherapy, a type of chemotherapy, immunotherapy, CRISPR-based DNA or RNA editing, etc.) based at least in part on the indication.

Then, the one or more of optional control modules 118 may instruct one or more of feedback modules 128 (such as feedback module 128-1) to generate a report about an individual associated with the sample (such a computer-aided diagnosis report with feedback, such as the cancer classification, the treatment recommendation, etc.). Furthermore, the one or more of optional control modules 118 may instruct one or more of communication modules 114 (such as communication module 114-1) to return, via network 120 and 122, outputs (such as the computer-aided diagnosis report, etc.) to computer 130 associated with a physician (such as a pathologist) or healthcare provider of the individual.

In these ways, computer system 100 may automatically and accurately assess the quality of samples associated with the one or more individuals. These capabilities may allow computer system 100 to reliably detect and diagnose a type of cancer in an automated manner. Moreover, the information determined by computer system 100 (such as the treatment recommendation, e.g., whether or not to perform a subsequent biopsy, radiation and/or a particular type of chemotherapy) may facilitate or enable improved use of existing treatments (such as precision medicine by selecting a correct medical intervention to treat a type of cancer, e.g., as a companion diagnostic for a prescription drug or a dose of a prescription drug) and/or improved new treatments. Consequently, the analysis techniques may facilitate accurate, value-added use of the measurement or test results, such as genetics analysis of a tissue biopsy sample or a liquid biopsy.

We now describe embodiments of the method. FIG. 2 presents a flow diagram illustrating an example of a method 200 for determining a sample-specific dynamic quality metric, which may be performed by a computer system (such as computer system 100 in FIG. 1). During operation, the computer system may receive information (operation 210) corresponding to a sample that is associated with a tissue biopsy or a liquid biopsy. Note that the information may include: a number of genetic molecules (such as nucleotides) associated with normal tissue, and a number of tumor genetic molecules associated with a tumor, which may include a number of mutated tumor genetic molecules. Moreover, the number of tumor genetic molecules (such as nucleotides) in the sample may correspond to a tumor fraction. Furthermore, the number of tumor genetic molecules in the sample may be based at least in part on histology, liquid biopsy data, pathology information, a simulation, or an output of a pretrained predictive model. In some embodiments, the mutated tumor genetic molecules may include: an SNV, a copy number variation, a fusion, an insertion, a deletion and/or an epigenetic change. Moreover, the genetic molecules may include DNA.

Then, the computer system may determine the sample-specific dynamic quality metric (operation 212) based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules. For example, the sample-specific dynamic quality metric may be a function of the first ratio or the second ratio when the number of genetic molecules is between 30 and 150. Note that, when the type of cancer includes lung cancer, the genomic region of interest may include chromosome 7, region p11.2 or a region that includes an EGFR. For example, for non-small cell lung cancer (NSCLC), the genomic regions of interest may include: EGFR exon 19 deletions L858R and T790M, EGFR exon 20 insertions, and/or KRAS G12C. Alternatively or additionally, when the type of cancer includes breast cancer, the genomic region of interest may include chromosome 6, region q25.1-q25.2 or a region that includes an ESR1.

Next, based at least in part on a comparison of the sample-specific dynamic quality metric and a threshold (operation 214), the computer system may selectively provide an indication (operation 216) whether a mutation or the type of cancer is present in the sample. Note that the threshold may be based at least in part on one or more of: the type of cancer, the sequencing coverage of one or more cancer-specific genomic targets, the sequencing coverage or tumor fraction (such as or corresponding to the first ratio or the second ratio). In some embodiments, the threshold may be based at least in part on the estimated tumor fraction of the sample.

In some embodiments, the computer system may optionally perform one or more additional operations (operation 218). For example, the computer system may provide one or more (or a combination of) treatment recommendations based at least in part on the indication.

Alternatively or additionally, the computer system may analyze the sample to determine genetic sequences, epigenetic data and/or a transcriptional state associated with the genetic molecules and the tumor genetic molecules. For example, the analysis may include whole exome sequencing or whole genome sequencing.

In some embodiments, receiving the information (operation 210) corresponding to the sample may include accessing, in memory associated with the computer system, the information corresponding to the sample.

In some embodiments of method 200, there may be additional or fewer operations. Furthermore, the order of the operations may be changed, and/or two or more operations may be combined into a single operation.

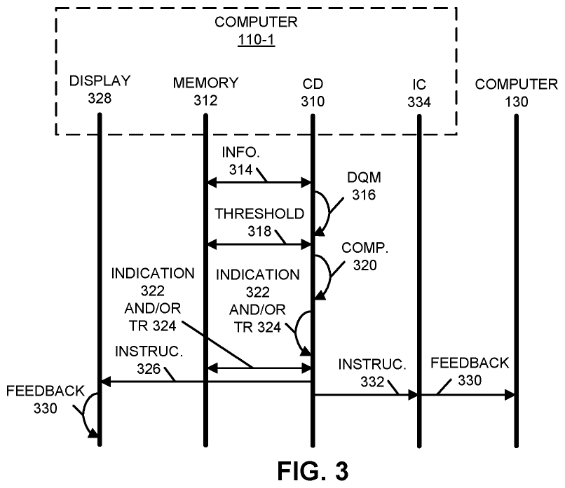

Embodiments of the analysis techniques are further illustrated in FIG. 3, which presents a drawing illustrating an example of communication among components in computer system 100. In FIG. 3, a computation device (CD) 310 (such as a processor or a GPU) in computer 110-1 may access, in memory 312 in computer 110-1, information 314 corresponding to a sample that is associated with a tissue biopsy or a liquid biopsy. Note that information 314 may include: a number of genetic molecules associated with normal tissue, and a number of tumor genetic molecules associated with a tumor, which may include a number of mutated tumor genetic molecules.

After receiving information 314, computation device 310 may determine a sample-specific dynamic quality metric (DQM) 316 based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules. Then, computation device 310 may compare 320 the sample-specific dynamic quality metric 316 to a threshold 318, which may be accessed in memory 312.

Moreover, based at least in part on the comparison 320, computation device 310 may compute an indication 322 whether a mutation or the type of cancer is present in the sample. Alternatively or additionally, computation device 310 may determine one or more (or a combination of) treatment recommendations (TR) 324 based at least in part on the indication 322.

After or while performing the preceding operations, computation device 310 may store results, including indication 322 and/or treatment recommendation 324, in memory 312. Next, computation device 310 may provide instructions 326 to a display 328 in computer 110-1 to display feedback 330, such as indication 322 and/or treatment recommendation 324 (and, more generally, a computer-aided diagnosis report). Alternatively or additionally, computation device 310 may provide instructions 332 to an interface circuit 334 in computer 110-1 to provide feedback 330 to another computer or electronic device, such as computer 130.

While FIG. 3 illustrates communication between components using unidirectional or bidirectional communication with lines having single arrows or double arrows, in general the communication in a given operation in this figure may involve unidirectional or bidirectional communication.

We now further describe embodiments of the analysis techniques. In general, sequencing coverage is nonuniform across a sample. Notably, the amount of sequencing coverage is dependent on the tumor fraction, and variant(s) found in different regions of the sequencing coverage can inform the needed coverage in other parts of the sample. Moreover, variability in current sequencing coverage may result in intermittent low sequencing coverage and failing a static quality-control threshold (which may result in up to an 80% failure rate). Furthermore, the high variability in sequencing coverage in assays often results in high numbers of coverage exceptions. The issues are not fully addressed by the use of higher DNA inputs and increased sequencing. Alternatively, samples with high tumor purity can be fully assayed using less sequencing/coverage.

In contrast with existing static quality-control approaches, in the disclosed analysis techniques a dynamic tumor purity threshold is used to ensure molecular detection with fewer sequencing reads. Moreover, a tumor-fraction-informed threshold in the analysis techniques may preserve clinical sensitivity and desired failure rates.

For example, the analysis techniques may involve a two-operation process. First, an estimate of the tumor purity may be computed. This may be performed in silico using an initial set of variant calls, via a pathologist assessment or using another analysis technique. Then, a threshold for a minimum number of genetic molecules needed to meet one or more performance metrics (such as an odds ratio) may be established. Notably, the threshold may be defined using linear scaling or may be computed probabilistically based at least in part on ratios of molecular qualities, such as double-strand overlaps from unique molecular identifier (UMI) barcoded molecules. In some embodiments, the threshold is predefined or precomputed. However, in other embodiments, the threshold is computed in real-time during the analysis techniques.

The analysis techniques may perform scaling from a tumor fraction to a sequencing coverage quality-control threshold, or may use tumor purity to indicate whether a tumor mutation burden (TMB) of a sample is evaluable. Note that a sequencing coverage quality-control metric may include or may be the median number of genetic molecules across probed regions, where only genetic molecules with multiple PCR duplicates or a second strand are counted.

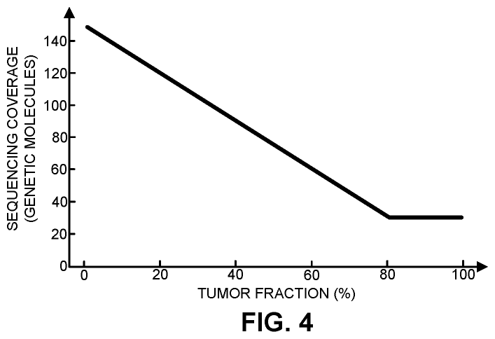

FIG. 4 presents a drawing illustrating an example of a dynamic quality-control threshold. Notably, the sequencing coverage quality-control threshold may scale linearly to a genetic-molecule floor or a minimum value of 30 genetic molecules. In some embodiments, the threshold may be defined as

$$150 \cdot ( 1 - ( \frac{tumor fraction}{100} ) ) ,$$

and when the tumor fraction is greater than or equal to 80%, the threshold equals 30. This threshold indicates that, at high tumor fraction, the amount of sequencing coverage is reduced. Note that, across the sample, the dynamic quality metric corresponding to the histology or liquid biopsy input (such as the number of tumor genetic molecules) may be compared to the threshold for a number of genetic molecules between 30 and 150. Moreover, the dynamic quality metric may include a tissue-specific component, such as genetic hotspot regions for a type of cancer. Thus, when it is estimated that there are insufficient tumor genetic molecules and/or when there are insufficient tumor genetic molecules in the sample, the sample may fail quality control and the indication or the test results may not be provided. Alternatively or additionally, when there is no or insufficient sequencing coverage in a genetic hotspot region for the type of cancer, the sample may fail quality control and the indication or the test results may not be provided.

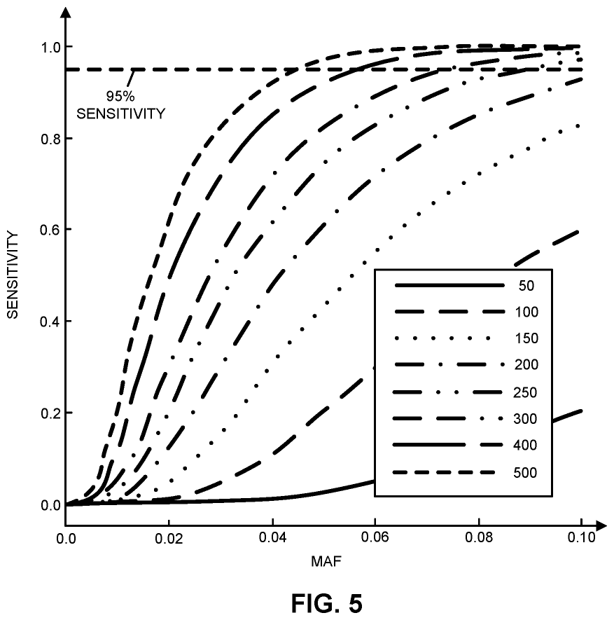

FIG. 5 presents a drawing illustrating an example of simulated sensitivity as a function of mutant allele frequency (MAF) for different sequencing coverage values. The sequencing coverage quality-control threshold may reach a floor of 30 genetic molecules when the tumor purity is greater than or equal to 80%.

Moreover, the tumor-fraction adjusted sequencing coverage quality-control threshold may reduce sample failures. Notably, in samples with high tumor purity, less sequencing may be required to recover genetic molecules from tumor DNA. Moreover, by scaling or adapting the sequencing coverage quality-control threshold with tumor purity, the sample pass rate may be increased without compromising sensitivity.

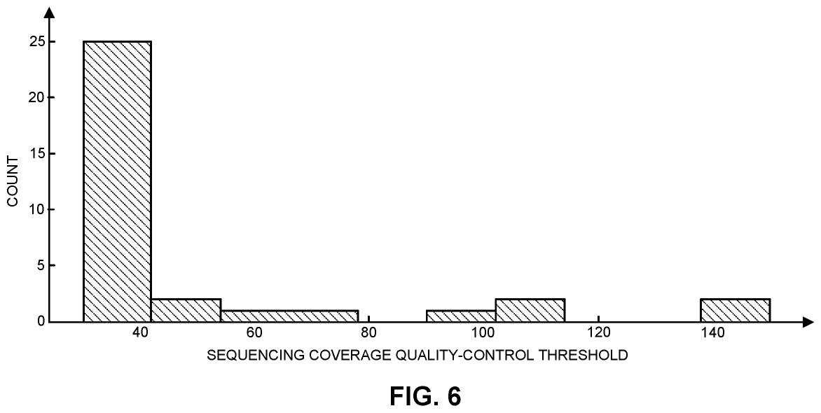

This is shown in FIG. 6, which presents a drawing illustrating an example of a tumor-fraction-adjusted threshold. In particular, data for 100 ng samples of extracted nucleic acid for 46 tumors was analyzed using a fixed and an adjustable threshold. With a fixed threshold of 150 genetic molecules, there were seven true positives and 39 false positives. However, with an adjustable threshold, there were 31 true positives and three false positives.

Given the high variability in sequencing coverage (both intra- and inter-sample), in combination with increased sequencing and input, the adaptive sequencing coverage quality-control threshold in the analysis techniques may reduce sequencing coverage failures. Consequently, an adjustable sequencing coverage quality-control threshold for exon failures and overall sequencing coverage requirements may reduce failure rates while ensuring SNV/indel detection.

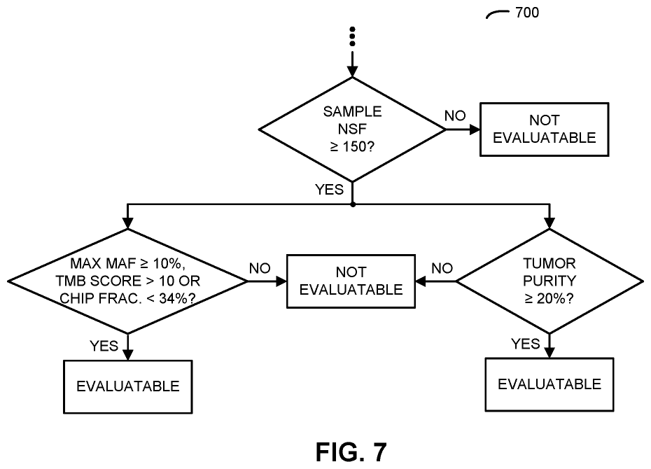

Furthermore, the use of the tumor fraction may provide benefits as to whether a tumor mutation burden of a sample is evaluable. Notably, the tumor mutation burden may be capable of being evaluated when the sample meets one or more criteria, such as: a maximum MAF (such as greater than or equal to 10%), a tumor mutation burden score (such as greater than 10%), a clonal hematopoiesis of indeterminate potential (CHIP) criterion (such as a CHIP fraction less than 34%) and/or a tumor purity criterion (such as greater than or equal to 20%). (Note that CHIP may refer to hematopoiesis in individuals that involves the expansion of hematopoietic stem cells that include one or more somatic mutations (e.g., hematologic cancer-associated mutations and/or non-cancer-associated mutations), but which otherwise lack diagnostic criteria for a hematologic malignancy, such as definitive morphologic evidence of dysplasia. CHIP is a common age-related phenomenon in which hematopoietic stem cells contribute to the formation of a genetically distinct subpopulation of blood cells.) This is shown in FIG. 7, which presents a flow diagram illustrating an example of a method 700 for dynamically analyzing the quality of a sample. Note that the tumor mutation burden evaluatable tumor purity cutoff of 20% may correspond to a heterozygous maximum MAF of 10%. Table 1 summarizes criteria that determine whether the tumor mutation burden of a sample is evaluatable based on the number of genetic molecules or the number of non-singleton families (NSF) in a sample (as shown in FIG. 7). The blank entries in Table I indicate that the value of a criterion does not matter or will not impact the evaluation status.

TABLE 1

| | Maximum | TMB | CHIP | Tumor | |
|---|---|---|---|---|---|
| Number | MAF ≥ | Score > | Fraction < | Purity ≥ | Evaluation |
| Genetic | Cutoff | Cutoff | Cutoff | Cutoff | Status |
| Molecules ≥ | | | | | |
| Threshold | | | | | |
| No | — | — | — | — | No |
| Yes | — | — | No | No | No |
| Yes | — | — | No | Yes | Yes |
| Yes | No | No | Yes | No | No |
| Yes | No | No | Yes | Yes | Yes |
| Yes | Yes | — | Yes | Yes | Yes |
| Yes | Yes | No | Yes | No or | Yes |
| | | | | Unknown | |
| Yes | No | Yes | Yes | — | Yes |

We now describe general features of the analysis techniques.

### Samples

While tissue biopsy and liquid biopsy are used as illustrations of a sample in the present disclosure, more generally a sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, and/or urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, e.g., a body fluid for analysis is plasma or serum containing cell-free nucleic acids, e.g., cell-free DNA. In some embodiments, the analysis techniques include obtaining the sample from a subject. Essentially any sample type is optionally utilized. In certain embodiments, e.g., the sample is tissue, blood, plasma, serum, sputum, urine, semen, vaginal fluid, feces, synovial fluid, spinal fluid, saliva, and/or the like. Typically, the subject is a mammalian subject (e.g., a human subject). In some embodiments, the sample is blood. In some embodiments, the sample is plasma. In some embodiments, the sample is serum.

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can include various amounts of nucleic acid. Typically, the amount of nucleic acid in a given sample is equates with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample includes nucleic acids from different sources, e.g., from cells and from cell-free sources (e.g., blood samples, etc.). Typically, a sample includes nucleic acids carrying mutations. For example, a sample optionally includes DNA carrying germline mutations and/or somatic mutations. Alternatively or additionally, a sample includes DNA carrying cancer-associated mutations (e.g., cancer-associated somatic mutations). In some embodiments, the sample includes cell-free DNA (i.e., cfDNA sample). In some embodiments, the cfDNA sample includes circulating tumor nucleic acids.

Exemplary amounts of cell-free nucleic acids in a sample before amplification typically range from about 1 femtogram (fg) to about 1 microgram (m), e.g., about 1 picogram (pg) to about 200 nanogram (ng), about 1 ng to about 100 ng, or about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, the analysis techniques include obtaining between about 1 fg to about 200 ng cell-free nucleic acid molecules from samples. In certain embodiments, the analysis techniques include obtaining between about 5 ng to about 30 ng of cell-free nucleic acid molecules from samples. In certain embodiments, the analysis techniques include obtaining between about 5 ng to about 100 ng of cell-free nucleic acid molecules from samples. In certain embodiments, the analysis techniques include obtaining between about 5 ng to about 150 ng of cell-free nucleic acid molecules from samples. In certain embodiments, the analysis techniques include obtaining between about 5 ng to about 200 ng of cell-free nucleic acid molecules from samples. In some embodiments, the amount is up to about 100 ng of cell-free nucleic acid molecules from samples. In some embodiments, the amount is up to about 150 ng of cell-free nucleic acid molecules from samples. In some embodiments, the amount is up to about 200 ng of cell-free nucleic acid molecules from samples. In some embodiments, the amount is up to about 250 ng of cell-free nucleic acid molecules from samples. In some embodiments, the amount is up to about 300 ng of cell-free nucleic acid molecules from samples. In some embodiments, the analysis techniques include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides in length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning operation in which cell-free nucleic acids, as found in solution, are separated from intact cells and other non-soluble components of the bodily fluid. In some of these embodiments, partitioning includes analysis techniques such as centrifugation or filtration. Alternatively, cells in bodily fluids are lysed, and cell-free and cellular nucleic acids processed together. Generally, after addition of buffers and wash operations, cell-free nucleic acids are precipitated with, e.g., an alcohol. In certain embodiments, additional clean-up operations are used, such as silica-based columns to remove contaminants or salts. Non-specific bulk carrier nucleic acids, e.g., are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis operations.

### Nucleic Acid Tags

In some embodiments, the nucleic acid molecules (from the sample of polynucleotides) may be tagged with sample indexes and/or molecular barcodes (referred to generally as ‘tags’). Tags may be incorporated into or otherwise joined to adapters by chemical synthesis, ligation (e.g., blunt-end ligation or sticky-end ligation), or overlap extension PCR, among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing operations are performed. In some embodiments, only the molecular barcodes are introduced prior to probe capturing and the sample indexes are introduced after sequence capturing operations are performed. In some embodiments, both the molecular barcodes and the sample indexes are introduced prior to performing probe-based capturing operations. In some embodiments, the sample indexes are introduced after sequence capturing operations are performed. In some embodiments, molecular barcodes are incorporated to the nucleic acid molecules (e.g., cfDNA molecules) in a sample through adapters via ligation (e.g., blunt-end ligation or sticky-end ligation). In some embodiments, sample indexes are incorporated to the nucleic acid molecules (e.g., cfDNA molecules) in a sample through overlap extension PCR. Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) portions corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcodes, or from about 5 to about 150 different molecular barcodes, or from about 20 to about 50 different molecular barcodes, ligated to both ends of a target molecule. Alternatively, from about 25 to about 1,000,000 different molecular barcodes may be used. For example, 20-50/20-50 molecular barcodes can be used. In some embodiments, 20-50 different molecular barcodes can be used. In some embodiments, 5-100 different molecular barcodes can be used. In some embodiments, 5-150 molecular barcodes can be used. In some embodiments, 5-200 different molecular barcodes can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, e.g., U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

### Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, e.g., in transcription mediated amplification. Other amplification exemplary methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular barcodes and/or sample indexes to a nucleic acid molecule using conventional nucleic acid amplification methods. The amplifications are typically conducted in one or more reaction mixtures. Molecular barcodes and sample indexes are optionally introduced simultaneously, or in any sequential order. In some embodiments, molecular barcodes and sample indexes are introduced prior to and/or after sequence capturing operations are performed. In some embodiments, only the molecular barcodes are introduced prior to probe capturing and the sample indexes are introduced after sequence capturing operations are performed. In certain embodiments, both the molecular barcodes and the sample indexes are introduced prior to performing probe-based capturing operations. In some embodiments, the sample indexes are introduced after sequence capturing operations are performed. Typically, sequence capturing protocols involve introducing a single-stranded nucleic acid molecule complementary to a targeted nucleic acid sequence, e.g., a coding sequence of a genomic region and mutation of such region is associated with a cancer type. Alternatively or additionally, typically the amplification reactions generate a plurality of non-uniquely or uniquely tagged nucleic acid amplicons with molecular barcodes and sample indexes at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

### Enrichment

Sequences can be enriched prior to sequencing. Enrichment can be performed for specific target regions or nonspecifically (‘target sequences’). In some embodiments, targeted regions of interest may be enriched with capture probes (‘baits’) selected for one or more bait set panels using a differential tiling and capture technique. A differential tiling and capture scheme uses bait sets of different relative concentrations to differentially tile (e.g., at different ‘resolutions’) across genomic regions associated with baits, subject to a set of constraints (e.g., sequencer constraints such as sequencing load, utility of each bait, etc.), and capture them at a desired level for downstream sequencing. These targeted genomic regions of interest may include natural or synthetic nucleotide sequences of the nucleic acid construct. In some embodiments, biotin-labeled beads with probes to one or more regions of interest can be used to capture target sequences, optionally followed by amplification of those regions, to enrich for the regions of interest.

Sequence capture may include the use of oligonucleotide probes that hybridize to the target sequence. A probe set strategy can involve tiling the probes across a region of interest. Such probes can be, e.g., about 60 to 120 bases long. The set can have a depth of about 2×, 3×, 4×, 5×, 6×, 8×, 9×, 10×, 15×, 20×, 50×, or more than 50×. The effectiveness of sequence capture depends, in part, on the length of the sequence in the target molecule that is complementary (or nearly complementary) to the sequence of the probe.

In some embodiments, the plurality of genomic regions includes genetic variants found in the Catalogue of Somatic Mutations in Cancer (,COSMIC), The Cancer Genome. Atlas (TCGA), or the Exome Aggregation Consortium (ExAC). In some cases, genetic variants may belong to a pre-defined set of clinically actionable variants. For example, such variants may be found in various databases of variants whose presence in a sample of a subject have been shown to correlate with or be indicative of a disease or disorder (e.g., cancer) in the subject. Such databases of variants may include, e.g., COSMIC, TCGA, and the ExAC. A pre-defined set of such catalogued variants may be designated for further bioinformatics analysis due to their relevance to clinical decision-making (e.g., diagnosis, prognosis, treatment selection, targeted treatment, treatment monitoring, monitoring for recurrence, etc.). Such a pre-defined set may be determined based on, e.g., analysis of clinical samples (e.g., of patient cohorts with known presence or absence of a disease or disorder) as well as annotation information from public databases and clinical literature.

### Sequencing

Sample nucleic acids flanked by adapters with or without poor amplification can be subject to sequencing. Sequencing methods include, e.g., Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (from Illumina), Digital Gene Expression (from Helicos BioSciences of Cambridge, Massachusetts), Next generation sequencing, Single Molecule Sequencing by Synthesis or SMSS (from Helicos), massively-parallel sequencing, Clonal Single Molecule Array (from Solexa, a division of Illumina, Inc. of San Diego, California), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

The sequencing reactions can be performed on one or more nucleic acid fragment types or regions known to contain markers of cancer or other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95% 99%, 99.9% or 100% of the genome. In other cases, sequence reactions may be performed on less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some cases, cell free polynucleotides may be sequenced with at least 1000,2000, 3000, 4000, 5000, 6000, 7000, 8000, 9000, 10000, 50000, or 100,000 sequencing reactions. In other cases, cell free polynucleotides may be sequenced with less than 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, or 100,000 sequencing reactions. In other cases, data analysis may be performed on less than 1000, 2000, 3000, 4000, 5000, 6000, 7000, 8000, 9000, 10000, 50000, or 100,000 sequencing reactions. An exemplary read depth is 1000-50000 reads per locus (base). In some embodiments, read depth can be greater than 50000 reads per locus (base).

### Analysis

Sequencing according to embodiments of the disclosed analysis techniques generates a plurality of sequencing reads or reads. Sequencing reads or reads according to the disclosed analysis techniques generally include sequences of nucleotide data less than about 150 bases in length, or less than about 90 bases in length. In certain embodiments, reads are between about 80 and about 90 bases, e.g., about 85 bases in length. In some embodiments, methods of the disclosed analysis techniques are applied to very short reads, i.e., less than about 50 or about 30 bases in length. Sequencing read data can include the sequence data as well as meta information. Sequence read data can be stored in any suitable file format including, e.g., VCF files, FASTA files or FASTQ files.

FASTA is originally a computer program for searching sequence databases and the name FASTA has come to also refer to a standard file format. See Pearson & Lipman, 1988, “Improved tools for biological sequence comparison,” PNAS 85:2444-2448. A sequence in FASTA format begins with a single-line description, followed by lines of sequence data. The description line is distinguished from the sequence data by a greater-than (‘>’) symbol in the first column. The word following the ‘>’ symbol is the identifier of the sequence, and the rest of the line is the description (both are optional). There should be no space between the ‘>’ and the first letter of the identifier. It is recommended that all lines of text be shorter than 80 characters. The sequence ends if another line starting with a ‘>’ appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer, as described by, e.g., Cock et al. (“The Sanger FASTQ file format for sequences with quality scores, and the Solexa/Illumina FASTQ variants,” Nucleic Acids Res 38(6): 1767-1771, 2009), which is hereby incorporated by reference in its entirety.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with ‘-’. In a preferred embodiment, the sequence data will use the A, T, C, G, and N characters, optionally including ‘-’ or U as-needed (e.g., to represent gaps or uracil).

In some embodiments, the at least one master sequence read file and the output file are stored as plain text files (e.g., using encoding such as ASCII; ISO/IEC; 646; EBCDIC; UTF-8; or UTF-16). A computer system provided by the disclosed. analysis techniques may include a text editor program capable of opening the plain text files. A text editor program may refer to a computer program capable of presenting contents of a text file (such as a plain text file) on a computer screen, allowing a human to edit the text (e.g., using a monitor, keyboard, and mouse) . Exemplary text editors include, without limit, Microsoft Word, macs, pico, vi. BBEdit, and TextWrangler. Preferably, the text editor program is capable of displaying the plain text files on a computer screen, showing the meta information and the sequence reads in a human-readable format (e.g., not binary encoded but instead using alphanumeric characters as they may be used in print human writing).

While methods have been discussed with reference to FASTA or FASTQ files, methods and systems of the disclosed analysis techniques may be used to compress any suitable sequence file format including, e.g., files in the Variant Call Format (VCF) format. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by Dapecek et al. (“The variant call format and VCFtools,” Bioinformatics 27(15):2156-2158, 2011), which is hereby incorporated by reference in its entirety. The header section may be treated as the meta information to write to the compressed files and the data section may be treated as the lines, each of which will be stored in a master file only if unique.

Certain embodiments of the disclosed analysis techniques provide for the assembly of sequencing reads. In assembly by alignment, e.g., the sequencing reads are aligned to each other or aligned to a reference sequence. By aligning each read, in turn to a reference genome, all of the reads are positioned in relationship to each other to create the assembly. In addition, aligning or mapping the sequencing read to a reference sequence can also be used to identify variant sequences within the sequencing read. Identifying variant sequences can be used in combination with the methods and. systems described herein to further aid in the diagnosis or prognosis of a disease or condition, or for guiding treatment decisions.

In some embodiments, any or all of the operations are automated. Alternatively, methods of the disclosed analysis techniques may be embodied wholly or partially in one or more dedicated programs, e.g., each optionally written in a compiled language such as C++ then compiled and distributed as a binary. Methods of the disclosed analysis techniques may be implemented wholly or in part as modules within, or by invoking functionality within, existing sequence analysis platforms. In certain embodiments, methods of the disclosed analysis techniques include a number of operations that are all invoked automatically responsive to a single starting cue (e.g., one or a combination of triggering events sourced from human activity, another computer program, or a machine). Thus, the disclosed analysis techniques provide methods in which any or the operations or any combination of the operations can occur automatically responsive to a cue. Automatically generally means without intervening human input, influence, or interaction (i.e., responsive only to original or pre-cue human activity).

The system also encompasses various forms of output, which includes an accurate and sensitive interpretation of the subject nucleic acid. The output of retrieval can be provided in the format of a computer file. In certain embodiments, the output is a FASTA file, FASTQ file, or VCF file. Output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact idiosyncratic Gapped Alignment Report (CIGAR) (Ning et al., Genome Research 11(10):1725-9, 2001, which is hereby incorporated by reference in its entirety). These strings are implemented, e.g., in the Exonerate sequence alignment software from the European Bioinformatics Institute (Hinxton, United Kingdom).

In some embodiments, a sequence alignment is produced (such as, e.g., a sequence alignment map or SAM, or binary alignment map or BAM file) including a CIGAR string (the SAM format is described, e.g., by Li et al., “The Sequence Alignment/Map format and SAMtools,” Bioinformatics. 25(16):207S-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string is useful for representing long (e.g., genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save some space), 3 matches, 2 deletions and 2 matches.

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a exonuclease activity in the presence of the nucleotides (e.g., A, C, G and T or U) in the form of dNTPs. Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including molecular barcodes, and the sequencing determines nucleic acid sequences as well as molecular barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation for e.g., sticky end ligation).

The nucleic acid sample is typically contacted with a sufficient number of adapters that there is a low probability (e.g., <1 or <0.1%) that any two copies of the same nucleic acid receive the same combination of adapter barcodes (i.e., molecular barcodes) from the adapters linked at both ends. The use of adapters in this manner permits identification of families of nucleic acid sequences with the same start and stop points on a reference nucleic acid and linked to the same combination of molecular barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent an analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, e.g., hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, :200, or 300 contiguous positions on a reference sequence, e.g., about 20-500, or about 50-300 contiguous positions.

Additional details regarding nucleic acid sequencing, including the formats and applications described herein are also provided in, e.g., Levy et al., Annual Review of Genomics and Human Genetics, 17: 95-115 (2014 Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microhiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7.482.120, 7,501,245, 6,818,395, 6,911,345, 7,501,245, 7,329,492, 7,170,050, 7,302,146, 7,313,308, and 7,476,503, which are each incorporated by reference in their entirety.

### Applications

### Cancer and Other Diseases

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease. Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gauther disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

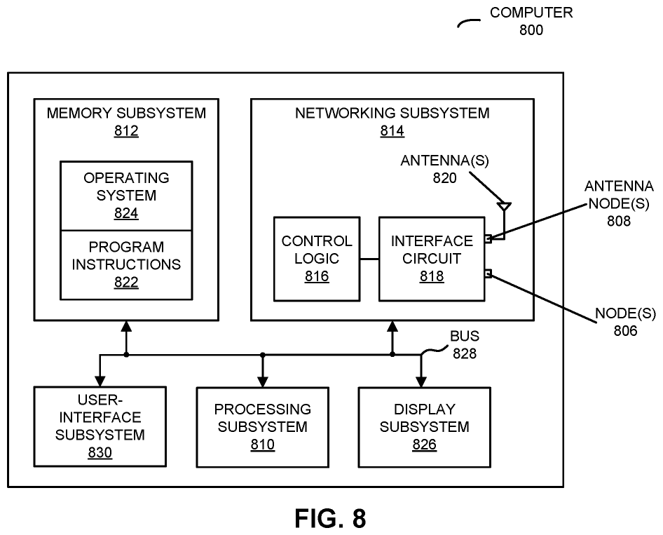

We now describe embodiments of a computer in a computer system, which may perform at least some of the operations in the analysis techniques. FIG. 8 presents a block diagram illustrating an example of a computer 800, e.g., in a computer system (such as computer system 100 in FIG. 1), in accordance with some embodiments. Computer 800 may regulate various aspects sample preparation, sequencing, and/or analysis, such as: determining the sample-specific dynamic quality metric, comparing the sample-specific dynamic quality metric to a threshold, and selectively providing an indication that a type of cancer is present in a sample. In some examples, computer 401 is configured to perform sample preparation and sample analysis, including nucleic acid sequencing.

Computer 800 may include: one of computers 110. This computer may include processing subsystem 810, memory subsystem 812, and networking subsystem 814. Processing subsystem 810 includes one or more devices configured to perform computational operations. For example, processing subsystem 810 can include one or more microprocessors (such as a single-core or a multi-core processor), ASICs, microcontrollers, programmable-logic devices, GPUs and/or one or more DSPs. Processing subsystem 810 may perform parallel processing of one or more operations in the analysis techniques. Note that a given component in processing subsystem 810 are sometimes referred to as a ‘computation device’.

Memory subsystem 812 includes one or more devices for storing data and/or instructions for processing subsystem 810 and networking subsystem 814. For example, memory subsystem 812 can include dynamic random access memory (DRAM), static random access memory (SRAM), flash and/or other types of memory. In some embodiments, instructions for processing subsystem 810 in memory subsystem 812 include: program instructions or sets of instructions (such as program instructions 822 or operating system 824), which may be executed by processing subsystem 810. Note that the one or more computer programs or program instructions may constitute a computer-program mechanism. Moreover, instructions in the various program instructions in memory subsystem 812 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 810. Thus, program instructions 822 may be precompiled for use with computer 800 or may be compiled at runtime. In some embodiments, program instructions 822 are stored or embodied on a type of non-transitory machine-readable medium, which may include a portable non-transitory machine-readable medium (e.g., a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, or any other medium from which a computer may read programming code and/or data).

In addition, memory subsystem 812 can include mechanisms for controlling access to the memory. In some embodiments, memory subsystem 812 includes a memory hierarchy that includes one or more caches coupled to a memory in computer 800. In some of these embodiments, one or more of the caches is located in processing sub system 810.

In some embodiments, memory subsystem 812 is coupled to one or more high-capacity mass-storage devices (not shown), which may be external to computer 800 and/or remotely located (and, thus, accessed via a network). For example, memory subsystem 812 can be coupled to a magnetic or optical drive, a solid-state drive, or another type of mass-storage device. In these embodiments, memory subsystem 812 can be used by computer 800 as fast-access storage for often-used data, while the mass-storage device is used to store less frequently used data. Note that data may be transferred from one location to another using, e.g., a network (such as the Internet and/or an intra-net) or physical data transfer (e.g., using a hard drive, thumb drive, or other data-storage device).

Networking subsystem 814 includes one or more devices configured to couple to and communicate on a wired and/or wireless network (i.e., to perform network operations), including: control logic 816, an interface circuit 818 and one or more antennas 820 (or antenna elements). (While FIG. 8 includes one or more antennas 820, in some embodiments computer 800 includes one or more nodes, such as antenna nodes 808, e.g., a metal pad or a connector, which can be coupled to the one or more antennas 820, or nodes 806, which can be coupled to a wired or optical connection or link. Thus, computer 800 may or may not include the one or more antennas 820. Note that the one or more nodes 806 and/or antenna nodes 808 may constitute input(s) to and/or output(s) from computer 800.) For example, networking subsystem 814 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, and/or another networking system.

Networking subsystem 814 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, computer 800 may use the mechanisms in networking subsystem 814 for performing simple wireless communication between electronic devices, e.g., transmitting advertising or beacon frames and/or scanning for advertising frames transmitted by other electronic devices.

Within computer 800, processing subsystem 810, memory subsystem 812, and networking subsystem 814 are coupled together using bus 828. Bus 828 may include an electrical, optical, and/or electro-optical connection that the subsystems can use to communicate commands and data among one another. Although only one bus 828 is shown for clarity, different embodiments can include a different number or configuration of electrical, optical, and/or electro-optical connections among the subsystems.

In some embodiments, computer 800 includes a display subsystem 826 for displaying information on a display, which may include a display driver and the display, such as a liquid-crystal display, a multi-touch touchscreen, etc. Moreover, computer 800 may include a user-interface subsystem 830, such as: a mouse, a keyboard, a trackpad, a stylus, a voice-recognition interface, and/or another human-machine interface. Note that user-interface subsystem 830 may include graphical user interface (GUI) and/or a web-based user interface

Additional details relating to computer systems and networks, data structures, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronet Database Systems: Design, Implementation, & Management, Cengage Learning, 11^{th}Ed. (2014), Tucker, Programming Languages, McGraw-Hill Science/Engineering/Math, 2nd Ed. (2006), and Kiloton, Cloud Computing Architected: Solution Design Handbook, Recursive Press (2011), each of which is hereby incorporated by reference in its entirety.

Computer 800 can be (or can be included in) any electronic device with at least one network interface. For example, computer 800 can be (or can be included in): a desktop computer, a laptop computer, a subnotebook/netbook, a server, a supercomputer, a tablet computer, a smartphone, a cellular telephone, a consumer-electronic device, a portable computing device, communication equipment, and/or another electronic device.

Although specific components are used to describe computer 800, in alternative embodiments, different components and/or subsystems may be present in computer 800. For example, computer 800 may include one or more additional processing subsystems, memory subsystems, networking subsystems, and/or display subsystems. Additionally, one or more of the subsystems may not be present in computer 800. Moreover, in some embodiments, computer 800 may include one or more additional subsystems that are not shown in FIG. 8. Also, although separate subsystems are shown in FIG. 8, in some embodiments some or all of a given subsystem or component can be integrated into one or more of the other subsystems or component(s) in computer 800. For example, in some embodiments program instructions 822 are included in operating system 824 and/or control logic 816 is included in interface circuit 818.

Moreover, the circuits and components in computer 800 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 814 and/or computer 800. The integrated circuit may include hardware and/or software mechanisms that are used for transmitting signals from computer 800 and receiving signals at computer 800 from other electronic devices. Aside from the mechanisms herein described, radios are generally known in the art and hence are not described in detail. In general, networking subsystem 814 and/or the integrated circuit may include one or more radios.

In some embodiments, an output of a process for designing the integrated circuit, or a portion of the integrated circuit, which includes one or more of the circuits described herein may be a computer-readable medium such as, e.g., a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the analysis techniques may be implemented using program instructions 822, operating system 824 (such as a driver for interface circuit 818) or in firmware in interface circuit 818. Thus, the analysis techniques may be implemented at runtime of program instructions 822. Alternatively or additionally, at least some of the operations in the analysis techniques may be implemented in a physical layer, such as hardware in interface circuit 818.

Note that the use of the phrases ‘capable of,’ ‘capable to,’ ‘operable to,’ or ‘configured to’ in one or more embodiments, refers to some apparatus, logic, hardware, and/or element designed in such a way to enable use of the apparatus, logic, hardware, and/or element in a specified manner.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the analysis techniques. In other embodiments, the numerical values can be modified or changed.

Moreover, as sequencing and liquid biopsy assays are changed (e.g., in sequencing depth and panels of common SNPs), methods and systems of the present disclosure may be modified as needed to obtain a set of applicable threshold values (e.g., one or more criteria/threshold to determine a dynamic quality metric of a sample).

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
an interface circuit;
a computation device coupled to the interface circuit; and
memory, coupled to the computation device, configured to store program instructions, wherein, when executed by the computation device, the program instructions cause the computer system to perform one or more operations comprising:
receiving information corresponding to a sample that is associated with a tissue biopsy or a liquid biopsy, wherein the sample comprises a number of genetic molecules associated with normal tissue, and a number of tumor genetic molecules associated with a tumor;
determining a sample-specific dynamic quality metric based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules; and
based at least in part on a comparison of the sample-specific dynamic quality metric and a threshold, selectively providing an indication of whether a mutation or the type of cancer is present in the sample.

2. The computer system of claim 1, wherein the mutation comprises: a single nucleotide variation (SNV), a copy number variation, a fusion, an insertion, a deletion or an epigenetic change.

3. The computer system of claim 1, wherein the genetic molecules comprise deoxyribonucleic acid (DNA).

4. The computer system of claim 1, wherein the one or more operations comprise providing one or more treatment recommendations based at least in part on the indication.

5. The computer system of claim 1, wherein the one or more operations comprise analyzing the sample to determine genetic sequences, epigenetic data or a transcriptional state associated with the genetic molecules and the tumor genetic molecules.

6. The computer system of claim 5, wherein the analysis comprises whole exome sequencing or whole genome sequencing.

7. The computer system of claim 1, wherein receiving the information corresponding to the sample comprises accessing, in the memory, the information corresponding to the sample.

8. The computer system of claim 1, wherein the first ratio or the second ratio corresponds to a tumor fraction.

9. The computer system of claim 1, wherein the number of tumor genetic molecules in the sample is based at least in part on histology, liquid biopsy data, pathology information, a simulation or an output of a pretrained predictive model.

10. The computer system of claim 1, wherein the sample-specific dynamic quality metric is a function of the first ratio or the second ratio when the number of genetic molecules is between 30 and 150.

11. The computer system of claim 1, wherein, when the type of cancer comprises lung cancer, the genomic region of interest comprises chromosome 7, region p11.2 or a region that comprises an epidermal growth factor receptor.

12. The computer system of claim 1, wherein, when the type of cancer comprises breast cancer, the genomic region of interest comprises chromosome 6, region q25.1-q25.2 or a region that comprises an estrogen receptor 1.

13. The computer system of claim 1, wherein the threshold is based at least in part on one or more of: the type of cancer, the sequencing coverage, or both.

14. A non-transitory computer-readable storage medium for use in conjunction with a computer system, the computer-readable storage medium configured to store program instructions that, when executed by the computer system, causes the computer system to perform one or more operations comprising:
receiving information corresponding to a sample that is associated with a tissue biopsy or a liquid biopsy, wherein the sample comprises a number of genetic molecules associated with normal tissue, and a number of tumor genetic molecules associated with a tumor;
determining a sample-specific dynamic quality metric based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules; and
based at least in part on a comparison of the sample-specific dynamic quality metric and a threshold, selectively providing an indication of whether a mutation or the type of cancer is present in the sample.

15. The non-transitory computer-readable storage medium of claim 14, wherein the sample-specific dynamic quality metric is a function of the first ratio or the second ratio when the number of genetic molecules is between 30 and 150.

16. The non-transitory computer-readable storage medium of claim 14, wherein the threshold is based at least in part on: the type of cancer, the sequencing coverage, or both.

17. A method for determining a sample-specific dynamic quality metric of a sample, comprising:
by a computer system:
receiving information corresponding to the sample that is associated with a tissue biopsy or a liquid biopsy, wherein the sample comprises a number of genetic molecules associated with normal tissue, and a number of tumor genetic molecules associated with a tumor;
determining the sample-specific dynamic quality metric based at least in part on: a type of cancer, sequencing coverage of one or more cancer-specific genomic targets associated with the type of cancer, and a first ratio of the number of tumor genetic molecules to a sum of the number tumor genetic molecules and the number of genetic molecules or a second ratio of the number of tumor genetic molecules to the number of genetic molecules; and
based at least in part on a comparison of the sample-specific dynamic quality metric and a threshold, selectively providing an indication of whether a mutation or the type of cancer is present in the sample.

18. The method of claim 17, wherein the number of tumor genetic molecules in the sample is based at least in part on histology, liquid biopsy data, pathology information, a simulation or an output of a pretrained predictive model.

19. The method of claim 17, wherein the sample-specific dynamic quality metric is a function of the first ratio or the second ratio when the number of genetic molecules is between 30 and 150.

20. The method of claim 17, wherein the threshold is based at least in part on: the type of cancer, the sequencing coverage, or both.
